(19) United States
(12) Patent Application Publication
(10) Pub. No.: US 2018/0103637 A1
(43) Pub. Date: Apr. 19, 2018

(54) NON-AQUEOUS SOLVENT SYSTEM FOR STERILANT COMPOSITIONS

(71) Applicant: American Sterilizer Company, Mentor, OH (US)

(72) Inventors: Phillip P. FRANCISKOVICH, Concord, OH (US); Kathleen A. FIX, Willoughby, OH (US); Donald G. ROSENHAMER, Medina, OH (US)

(21) Appl. No.: 15/296,163

(22) Filed: Oct. 18, 2016

(51) Int. Cl. A01N 37/12; A61L 2/18; A01N 25/02

(52) U.S. Cl. CPC A01N 37/12; A61L 2202/24; A01N 25/02; A61L 2/18

(57) ABSTRACT

A sterilant composition is disclosed which contains peracetic acid and a non-aqueous solvent. The sterilant composition may include one or more of a buffer, an anticorrosive agent and a chelator. The sterilant composition may be free of added water. The sterilant composition may be used for sterilizing articles such as medical, dental, pharmaceutical, veterinary or mortuary instruments, devices, and the like.

### TECHNICAL FIELD

This invention relates to sterilant compositions suitable for sterilizing articles such as medical, dental, pharmaceutical, veterinary or mortuary instruments, devices, and the like, the sterilant compositions including a non-aqueous solvent. This sterilant composition also may be referred to as a sterilant or as a sterilant mixture.

### BACKGROUND

Medical, dental, pharmaceutical, veterinary or mortuary instruments and devices that are exposed to blood or other bodily fluids require sterilizing or disinfecting between each use. Aqueous-based liquid sterilizing or disinfecting systems are typically used to clean and decontaminate instruments and devices that cannot withstand the high temperatures of steam sterilization. These aqueous-based sterilizing systems are effective in sterilizing instruments but introduce water-based hazards and additional formulation complexities such as the need to buffer the solution and the need to manage the broadly variable water hardness chemistries encountered when using municipal water sources to dilute the sterilizing and disinfecting concentrates at their point of use. In addition, the presence of water may require prolonged drying times, or can, in the opinions of some experts, lead to recontamination of wet instruments from environmental sources.

What is needed are non-aqueous and/or organic solvent-based sterilant delivery media which can be substituted for the water intentionally added in water-based sterilant compositions and in the delivery and dispersal of active sterilant or disinfectant chemistries.

### SUMMARY

In one embodiment, the present invention relates to a sterilant composition consisting essentially of peracetic acid and a non-aqueous solvent. In one such embodiment, the peracetic acid is commercially available peracetic acid, which contains the peracetic acid, acetic acid, hydrogen peroxide, minimal water, and sulfuric acid. In this embodiment, the only water present is the water present in the commercially available peracetic acid. In another embodiment, the only water present is the water formed in the reaction used to form peracetic acid in the non-aqueous solvent. In this embodiment, no additional water is purposely added to the sterilant composition, so that the composition only contains water that was included in the commercially available peracetic acid concentrate.

In one embodiment in which the sterilant composition consists essentially of peracetic acid and a non-aqueous solvent, the peracetic acid is formed or made in the original or another non-aqueous solvent, and the sterilant composition contains essentially no water, other than possibly small quantities of water formed as a by-product in the peracetic acid-forming reaction, and the sterilant composition contains no other added water. Depending on the method of making the peracetic acid, there may be by-products that would be a part of the final sterilant composition. For example, one method of making peracetic acid involves catalytic oxidation of acetaldehyde, and forms acetic acid as a by-product. In this case, the sterilant composition would include the acetic acid by-product, within the “consisting essentially” term.

In one embodiment, the present invention relates to a sterilant composition consisting essentially of peracetic acid, water (small amounts intentionally added to aid in the dilution of the concentrated peracetic acid component) and a non-aqueous solvent. In this embodiment, the non-aqueous solvent is present in a concentration of at least about 40% by weight, or at least about 50% by weight, or at least about 60% by weight, based on the overall sterilant composition. In one embodiment, the peracetic acid in this sterilant composition is commercially available peracetic acid. In one embodiment, this sterilant composition contains no added water, other than that present in the peracetic acid.

It is noted that commercially available peracetic acid may be provided as a 40% solution in acetic acid having the following approximate composition by weight: peracetic acid, 40-42%; H₂O₂, 4-5%; acetic acid, 37-40%; H₂SO₄, 0.5-1%; water, 10-14%. In some embodiment, the sterilant composition may contain a stabilizer. This form of peracetic acid is obtained via the usual industrial method of making peracetic acid, which involves reaction of acetic acid with hydrogen peroxide in the presence of sulfuric acid.

In one embodiment, the present invention relates to a sterilant composition comprising, consisting essentially of, or consisting of, (A) an anti-microbial agent comprising, consisting essentially of, or consisting of, peracetic acid; and (B) a reagent mixture comprising, consisting essentially of, or consisting of, a buffer, an anticorrosive agent and a chelator; and (C) a non-aqueous solvent. In one embodiment, the sterilant composition is characterized by the absence of added water, other than that present in the peracetic acid. This sterilant composition may be dispersed or dissolved in a preponderance or major amount of the non-aqueous solvent to form a sterilant composition, which may be referred to as a sterilant composition mixture or a sterilizing medium. When used in a sterilizing process, components (A) and (B) may be supplied separately and dispersed in the non-aqueous solvent (C), either simultaneously or sequentially, at the time the sterilization process is conducted. In this embodiment, the sterilant mixture may contain a small amount of water, either as a component of the peracetic acid, or, in other embodiments, water intentionally added.

In one embodiment, the sterilant composition consists essentially of (A) an anti-microbial agent comprising peracetic acid; and (C) a non-aqueous solvent. In one embodiment, this sterilant composition is characterized by the absence of added water. In one embodiment, component (A) consists essentially of peracetic acid, thus forming a sterilant composition consisting essentially of peracetic acid and a non-aqueous solvent. In this embodiment, due to inclusion of the non-aqueous solvent (C) and the substantial absence of water, there is no need for the component (B), the reagent mixture containing a buffer, an anticorrosive agent and a chelator.

In one embodiment, the sterilant composition, consists essentially of peracetic acid; and a non-aqueous solvent. In one embodiment, the sterilant composition consists essentially of the peracetic acid, the non-aqueous solvent, and one or more of a buffer, an anticorrosive agent and a chelator. In one embodiment, the peracetic acid is a commercially available peracetic acid which consists essentially of the peracetic acid, acetic acid, hydrogen peroxide, sulfuric acid and a minor amount of water, the non-aqueous solvent present in a major amount in the sterilant composition. In one embodiment, the composition is free of separately added water. In one embodiment, the buffer comprises an alkali metal phosphate, an alkali metal carbonate, or a mixture thereof. In one embodiment, the anticorrosive agent comprises benzotriazole, tolyltriazole, a sodium salt of benzotriazole, a sodium salt of tolyltriazole, or a mixture of two or more thereof. In one embodiment, the chelator comprises ethylenediaminetetraacetic acid, hydroxyethylidenediphosphonic acid, a sodium salt of ethylenediaminetetraacetic acid, a sodium salt of hydroxyethylidenediphosphonic acid, or a mixture of two or more thereof. In one embodiment, the peracetic acid is dispersed in the non-aqueous solvent to form the sterilant composition, the concentration of peracetic acid in the non-aqueous solvent being in the range from about 0.1 to about 10 grams per liter. In one embodiment, the sterilant composition is free of water.

These embodiments of the present invention include the significant advantage of a very simple sterilant composition for use as a sterilizing agent, containing only peracetic acid and a non-aqueous solvent, or containing only peracetic acid, all or part of the reagent mixture (B) and the non-aqueous solvent. These embodiments also have the advantage of reducing or eliminating the need for water hardness controls, water-based corrosion controls, buffering, drying times sufficient for water to dry, and the risk of environmental recontamination of articles that are still wet from exposure to prior art systems that are comprised predominately of water as solvent.

This invention also relates to a process for sterilizing an article comprising contacting the article with the foregoing sterilant composition embodiments. This process may be conducted in a sterilizing apparatus, the sterilizing apparatus comprising a sterilization chamber and a sterilant introduction system, the process comprising: placing the article in the sterilization chamber; providing the sterilization chamber with one of the sterilant composition embodiment(s) described above; spraying, flowing, immersing or otherwise bringing the sterilant composition in the sterilization chamber into contact with the article for an effective period of time, and at an effective temperature to sterilize the article; draining the thus-used sterilant composition from the sterilization chamber; flowing a rinse of a non-aqueous solvent into the sterilization chamber to bring it into contact with the article; and, optionally, rinsing with water or another, more volatile and/or safer non-aqueous solvent, and then removing the article from the sterilization chamber.

In one embodiment, the process for sterilizing an article includes contacting the article with the sterilant composition of any of the preceding claims. In one embodiment, the process is applied to an article made of a material comprising brass, copper, aluminum, stainless steel, carbon steel, plastic, glass, adhesive or a combination of two or more thereof. In one embodiment, the process is applied to an article such as a medical, dental, pharmaceutical, veterinary or mortuary instrument or device. In one embodiment, the process is carried out with the temperature of the sterilant composition in the range from about 20 to about 80° C., or from about 40° C. to about 60° C. In one embodiment, the process is carried out with an exposure time of the article to the sterilant composition in the range from about 0.5 to about 240 minutes, or from about 2 to about 60 minutes.

### DETAILED DESCRIPTION

All ranges and ratio limits disclosed in the specification and claims may be combined, e.g., the upper limit of one embodiment range may be combined with the lower limit of another embodiment range, to form a new range for the given component. It is to be understood that unless specifically indicated, references to “a,” “an” and/or “the” may include one or more than one, and that reference to an item in the singular may also include the item in the plural.

The phrase “and/or” should be understood to mean “either one or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

The transitional words or phrase “consisting essentially of” is to be understood to be closed to any ingredient that would affect the basic and novel characteristics of the components listed under these transitional words or phrase, and this means that, any ingredient that would affect how the sterilant composition disclosed herein functions, is excluded by the term “consisting essentially of.” When “consisting essentially of” is used herein, it excludes additional components that might be added to the sterilant mixture that would inhibit the function of the sterilant in its ability to kill spores or other microorganism forms, that would increase or decrease the activity of a component of a mixture disclosed or used with this term, or that affects the function of the sterilant composition as a whole, compared to the same sterilant composition without the added component. Thus, the term “consists essentially of,” as used herein, is used in the standard patent sense, i.e., in which nothing else is included that effects the way the sterilant composition functions, or in which nothing else is included that affect the basic and novel aspects of the invention. Thus, the sterilant composition may contain additional ingredients, for example, a minor amount of water, that does not affect how the sterilant composition functions. Examples of ingredients that would be expected to affect how the sterilant composition functions include enzymes, additional sterilizing agents beyond the peracetic acid and the hydrogen peroxide that is present in some embodiments, antifoam agents, molybdate ions, and surfactants.

The transitional term “consisting of” is to be understood to close the composition so described to any ingredient other than those specifically included.

The term, “a minor amount” applied to an ingredient of the sterilant composition, for example, applied to water in the sterilant composition, means that the sterilant composition contains 10% or less, or 7% or less, or 5% or less, or 2% or less, or 1% or less of the specified ingredient such as water. As noted, in at least some embodiments of the present invention, water is not purposefully added.

The term “killing” or “kill” with respect to spores refers to rendering the spores incapable of reproduction, metabolism and/or growth.

The term “log reduction” is a mathematical term to show the number of live spores killed by contacting the spores with the sterilant composition of the invention. A “4 log reduction” means that the number of live spores is 10,000 times smaller. A “5 log reduction” means that the number of live spores is 100,000 times smaller. A “6 log reduction” means that the number of live spores is 1,000,000 times smaller.

The term “sterilization” refers to rendering a substance incapable of reproduction, metabolism and/or growth. The term “sterilization” includes microbial deactivation. While sterilization is often taken to refer to a total absence of living organisms, the term may be used herein to refer to a substance free from living organisms to a degree agreed to be acceptable. Unless otherwise indicated, the term “sterilization” may be used herein to also refer to processes less rigorous than sterilization, for example, disinfection, sanitization, decontamination, cleaning, and the like. Variations of the term “sterilization,” such as sterilant, sterilizing, etc., may also be used herein to refer to and encompass related variants associated with sterilization processes as well as processes less rigorous than sterilization (e.g., disinfectant, disinfecting, etc.).

The inventive sterilant composition may comprise, consist essentially of, or consist of, a sterilant composition which may be made by dispersing or dissolving components (A) and (B) in (C) a non-aqueous solvent. In one embodiment, component (B) is omitted. In one embodiment, the non-aqueous solvent may be an alcohol, a nitrile, a ketone, an ester, an alkyl halide, a terpene, or an organic carbonate.

In one embodiment, the non-aqueous solvent is an alcohol. Alcohols include the lower alcohols (1-3 carbon atoms, e.g. methanol, ethanol and 1-propanol, 2-propanol) as well as the higher alcohols (4-10 carbons as exemplified by 1-butanol, 2-butanol, 2-methanol-1-propanol, 2-methanol-2-propanol, 1-pentanol, etc.). These alcohols are presented as representative and other alcohols known to those skilled in the sterilization arts are contemplated, including those substituting other side groups such as, for example, the phenyl group, —C₆H₅.

In one embodiment, the non-aqueous solvent includes liquid polyalkyleneglycols, including polyethylene glycol and polypropylene glycol. As known in the art, the liquidity and viscosity of such compounds depends on the molecular weight, and this can be determined by the skilled person as needed to provide the desired sterilant composition characteristics.

In one embodiment, the non-aqueous solvent includes liquid alkyl polyalkylene glycol ethers, i.e., alkyl polyethylene and/or polypropylene glycol ethers, which correspond to the following formula (I):

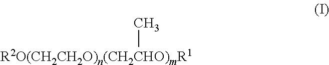

wherein each of R¹ and R² are, independently, H or C₁-C₆ alkyl, linear or branched, provided that at least one of R¹ and R² is not H, and wherein m and n range, in one embodiment, from 1-20, and in another embodiment from 2 to 10. These are known, commercially available compounds.

Suitable non-aqueous solvents include nitrile compounds, such as acetonitrile, esters, such as ethyl acetate, ethers, such as tetrahydrofuran, and low boiling alkyl halides, such as chloroform or tetrachloroethylene.

Suitable non-aqueous solvents include low molecular weight ketones, such as acetone or methyl ethyl ketone; terpenes, such as geraniol, terpineol, limonene, linalool, farnesene, and pinene; organic carbonates such as propylene carbonate; glycols, including ethylene glycol, propylene glycol, diethylene glycol; and aldehydes, such as propionaldehyde, crotonaldehyde, butyraldehyde.

Suitable non-aqueous solvents include nitrogen-containing compounds, such as amines or amides, which may also be suitable on their own as disinfection agents.

In one embodiment, the non-aqueous solvent does not include any of methyl acetate, ethyl acetate, or acetone, and in one embodiment, the non-aqueous solvent does not include any organic solvent containing a phosphate, such as a trialkylester of phosphoric acid, in which the alkyl groups are independently C₁-C₁₀ branched or unbranched alkyl.

The foregoing categories of non-aqueous solvents, and particularly the alcohols, glycols and polyalkyleneglycols mentioned above, may be used in any combination of two or more of the foregoing non-aqueous solvents. In one embodiment, the disclosed non-aqueous solvent(s) may be provided with or without a minor amount of water included as an impurity or minor component.

Sterilant compositions containing non-aqueous solvents of these types may provide, inter alia, the added benefits of solubilizing organic matter (soils commonly encountered on used instruments), penetration into recessed areas, easier and more complete film forming for enhanced contact between the sterilant chemistry (e.g., avoiding the “beading” or non-wetting effects observed as a result of the surface tension of water) and the device being processed, and drying of the processed devices and sterilization apparatus more quickly than an aqueous solution alone because of the generally greater vapor pressure and volatility of many of the non-aqueous solvents relative to water.

Rinsing may be performed using the same non-aqueous solvent, and then drying with either purges with dry air or by evaporation under partial pressure or vacuum. In some embodiments, the non-aqueous solvent may be removed from the items sterilized by rinsing with water or a more-volatile and/or safer non-aqueous solvent.

By substituting some or all of the non-aqueous solvents disclosed above for the conventional water, new benefits are attained. These benefits are exemplified by but not limited to the following:

Water has a well defined surface tension that sometimes needs to be modulated with the addition of filming and/or rinsing agents in the builders portion of the composite use dilution. By substituting alcohols or any of the other non-aqueous solvents or mixtures thereof for water the surface tension may be reduced sufficiently to reduce or eliminate the need for these modulators.

Water, from the most common sources, also contains dissolved components, e.g., hardness chemicals, which have to be considered when constructing aqueous-based sterilant or disinfectant chemistries. These components do not exist in any of the non-aqueous solvents or dispersants described above. The absence of such dissolved components would reduce or eliminate the need for hardness control, especially if these non-aqueous solvents are used for both processing and rinsing.

Water has a relatively low vapor pressure and thus tends to remain on processed devices longer at atmospheric pressure than would many of those liquid solvents described above. It has been argued that this remaining water traps and retains contaminating organisms after the device has been processed. Current methods often call for a final purge with isopropanol to drive off remaining water.

As is well known, water can be corrosive. The disclosed non-aqueous solvents are either much less or not corrosive, depending, of course, on the specific non-aqueous solvent and the specific substrate with which the solvent may come into contact. This could eliminate the need for an anti-corrosive in the use dilution of a non-water based system.

Water can cause swelling in some polymeric materials that are part of the construction of many medical devices. One may chose from the alternate dispersants above for their relative differences in causing this effect.

Water is often repulsed from common components of the soils found on used devices, so much so that it is common practice to include surfactants and other ingredients to make water better at penetration, filming and dissolution of the fatty or proteinaceous components of soils and blood. Using a non-aqueous solvent could eliminate the need for any surface active ingredients from the formulated chemistry.

Water contributes to foaming. Many of the dispersants mentioned here do not allow foaming. This would reduce or eliminate the need for anti foaming reagents that are sometimes configured into water based systems.

Some of the disclosed non-aqueous solvents are relatively neutral with respect to their individual effect on organisms (i.e., are not themselves effective in sterilizing or disinfecting), while others have natural disinfecting properties (e.g., the lower alcohols). Some alcohols dehydrate proteins, for example, to such a degree that the proteins are denatured. This in itself would kill those organisms whose essential proteins are so denatured.

Benefits include the reduction, or elimination, of many of the problems associated with the exclusive use of water. Additional benefits include the introduction of properties not found in water for heightened performance of a sterilizing or disinfecting system, e.g., better dispersion, better filming, better penetration, better sterilization and/or disinfection, better rinsing and faster drying, fewer hardness issues, less likelihood for rapid recontamination from the environment, lesser needs for surfactants, chelants, filming and rinsing agents, and better (or different) extraction of soils and bio-matter.

Due to the physical and chemical nature of some of the non-aqueous solvents disclosed herein, it becomes possible to consider means to recycle and purify used materials by: distillation, chromatographic separation, phase separation and similar methods so that the used non-aqueous solvents can be reclaimed, re-purified and reused in multi-use applications.

In one embodiment, component (A) may contain peracetic acid and optionally one or more additional anti-microbial agents. As noted above for the embodiments containing only peracetic acid, the peracetic acid in component (A) may further comprise acetic acid, hydrogen peroxide, sulfuric acid and a minor amount of water. In one embodiment, the water is present only as a component of the peracetic acid, and is not purposefully added simply as water.

Component (A), in another embodiment, consists essentially of peracetic acid and does not contain any other anti-microbial agent. Component (A), in another embodiment, consists essentially of peracetic acid, hydrogen peroxide, sulfuric acid and a minor amount of water, and does not contain any other anti-microbial agent. As noted, in embodiments of the invention, no water is purposely added to the sterilant composition, and the only water present is water that is necessarily present in one or more of the components of the sterilant composition.

Component (B), when present, may comprise a builder formulation, which may be used in combination with more dilute embodiments of component (A) to provide for buffering capability (pH modulation), anticorrosive properties, and chelation capacity (water softening or metal ion sequestration) for those embodiments of the present invention that include more than trace amounts of water, or added water. Component (B) may comprise a buffer, an anticorrosive agent and a chelator. Component (B), when present, may consist essentially of a buffer, an anticorrosive agent and a chelator. Component (B), when present, may consist of a buffer, an anticorrosive agent and a chelator. The ingredients of the component (B) should be soluble in the non-aqueous solvent. In one embodiment, water is only present as a component of the buffer, anticorrosive agent and/or chelator.

In one embodiment, it is preferred that all of the components of both component A and component (B) be added to the non-aqueous solvent in the absence of added water. Concentrations of the components of component (B) depend, to some extent, upon the specific molecule being used for the purpose, and can be suitably determined by the skilled person.

The buffer may comprise an alkali metal phosphate, an alkali metal carbonate, or a mixture thereof. The alkali metal may comprise sodium or potassium. The buffer may comprise one or more of monosodium phosphate, disodium phosphate, trisodium phosphate, monopotassium phosphate, dipotassium phosphate, tripotassium phosphate, sodium carbonate, or a mixture of two or more thereof. Disodium phosphate may be preferred. The buffer may be partially soluble in the non-aqueous solvent. However, since a buffer is normally only needed when water is present, when no water is present, no buffer is needed.

Suitable buffer compounds include glycine and related compounds, including bicine (2-(Bis(2-hydroxyethyl)amino)acetic acid), tricine (N-(2-Hydroxy-1,1-bis(hydroxymethyl)ethyl)glycine), glycylglycine (2-[(2-Aminoacetyl)amino]acetic acid), amines, including MOPS, PIPES, MES, CAPS, TRIS, TAPSO, TAPS, HEPES, TES, imidazoline, citrate, succinate and salicylate salts, in which the counterion may include the alkali metals, alkaline earth metals, polycarboxylic acid and salts thereof with the alkali metals and/or alkaline earth metals, and combinations thereof. Other suitable buffers may be found by referring to the Buffer Reference Center, provided by Sigma-Aldrich on its website. The buffer concentration usually ranges from about 10 ppm to about 10000 ppm, or from about 100 ppm to about 1000 ppm, based on the water present in the system. Where no or substantially no water is present, the buffer can be present at a concentration in the range from about 1 ppm to about 1000 ppm, or from about 10 ppm to about 100 ppm, based on the entire sterilant composition.

The anticorrosive agent may comprise benzotriazole, tolyltriazole, or a mixture thereof. Benzotriazole may be preferred. Other suitable anti-corrosive agents include known anticorrosive agents, including aliphatic and aromatic quaternary cyclic and acyclic amines, amine phosphate esters, amine oxides, alkyl polyglucosides, amine phosphates, polycarboxylic acids, imidazolines or their salts, amines such as morpholine, ammonia, piperazine, MOPA; ethylene di-, tri-, or tetra-amine; ethanolamine, TEA, diethylhydroxylamine, ethylamine, triisopropylamine and/or their salts. An anti corrosive for copper is HPGCP. The anticorrosive agent concentration usually ranges from about 10 ppm to about 1000 ppm, or from about 50 ppm to about 500 ppm, based on the water present in the system. Where no or substantially no water is present, the anticorrosive agent can be present at a concentration in the range from about 1 ppm to about 1000 ppm, or from about 10 ppm to about 100 ppm, based on the entire sterilant composition.

Specific examples of corrosion inhibitors include but are not limited to: 5 mM 4-methyl-1-(p-tolyl)-imidazole (TMI); 10 mM 1-phenyl-4-methyl-imidazole (PMI); 10 mM (MAcT) or 1 mM 2-mercapto 5-R-amino-1,3,4 thiadiazole (MAT); and 1 mM benzotriazole (BTA).

The chelator may comprise ethylenediaminetetraacetic acid, hydroxyethylidenediphosphonic acid, or a mixture thereof. A preferred chelator may be ethylenediaminetetraacetic acid. Other suitable chelators include, for example, crown ethers, cryptands and fullerenes. The chelator should be soluble in the non-aqueous solvent. The chelator concentration usually ranges from about 10 ppm to about 1000 ppm, or from about 50 ppm to about 500 ppm, based on the water present in the system. Where no or substantially no water is present, the chelator can be present at a concentration in the range from about 1 ppm to about 1000 ppm, or from about 10 to about 100 ppm, based on the entire sterilant composition.

A chelator may only be needed in the sterilant compositions that have a significant water component. Any of the standard chelators for aqueous solutions noted above and/or known to those familiar with the art could be used—with the proviso that they should remain functionally dissolved in the sterilant composition and at a concentration sufficient to handle the hardness of the water portion of the mixture.

In one embodiment, component (B) may comprise: disodium phosphate; benzotriazole; and ethylenediaminetetraacetic acid as the three components.

Herein, when a component is disclosed as present in a weight percentage, e.g., 10% by weight, the percentage is of the specific sterilant composition being referred to, unless otherwise stated. Thus, where a component is described as a percentage by weight of component (A), it is based on the component (A) only. Where a component is described as a percentage by weight of the sterilant composition, it is based on the total sterilant composition, including all components.

In one embodiment, the non-aqueous solvent in the overall sterilant composition may range from about 40% to about 98% by weight, and in one embodiment, from about 80% to about 95% by weight, or from about 85% to about 95% by weight of the overall sterilant composition.

A peracetic acid solution which may be used as component (A) is commercially available from Sigma-Aldrich. This solution is believed to contain 39% by weight peracetic acid, 45% by weight acetic acid, 6% by weight hydrogen peroxide, and about 10% or less free water, and also contains sulfuric acid. This commercially available peracetic acid solution may be dispersed as appropriate in the non-aqueous solvent.

In one embodiment, the peracetic acid is prepared in a non-aqueous solvent. A method of making peracetic acid in an organic solvent is disclosed in U.S. Pat. No. 4,904,821. This patent describes the preparation of peracetic acid in an organic solvent containing a phosphate, such as a trialkylester of phosphoric acid, in which the alkyl groups are independently C₁-C₁₀ branched or unbranched alkyl. Other substituents replacing these alkyl groups include tricyclohexyl, triphenyl, tricresyl, diphenylcresyl. Trimethyl, triethyl, tributyl and trioctyl phosphates are disclosed as being preferred, in the '821 patent. This patent further discloses the transfer of the peracetic acid from the phosphoric ester solvent into another organic solvent, such as, e.g., aliphatic esters, such as methyl to propyl acetates, which are preferred, aromatic hydrocarbons, e.g., chlorinated hydrocarbons, including a mixture with the esters, can also be used. Another process for preparing peracetic acid in an organic solvent is disclosed in U.S. Pat. No. 2,830,080, which uses methyl acetate, ethyl acetate, acetone or acetic acid as solvent, and treats acetaldehyde with oxygen in the presence of a catalyst to form peracetic acid in these specific solvent, with acetic acid being the preferred solvent. Further details may be obtained from a review of U.S. Pat. Nos. 2,830,080 and 4,904,821, which are incorporated herein by reference in their entireties. In one embodiment, the non-aqueous solvent of the present invention is a solvent other than the solvents disclosed in these patents.

In one embodiment, the sterilant composition contains a concentration of peracetic acid in the non-aqueous solvent in the range from about 0.5 to about 10 grams per liter, or from about 1.2 to about 3.5 grams per liter, based on the total volume of the sterilant composition.

When present, the concentration of component (B) may be in the range from about 3 to about 18 grams per liter, or from about 5 to about 15 grams per liter, based on the total volume of water present in the sterilant composition. Small amounts of water only need a small amount of component (B). Thus, in one embodiment, the amount of component (B) is based on the actual quantity of water present in the sterilant composition. For example, if 100 ml of the sterilant composition contains 10 ml of water, and component (B) is present in the water at a concentration of 5 g/I, the 10 ml of water (and the 100 ml total volume) will contain 0.05 g of component (B).

As used herein, the “use” concentration of the peracetic acid is that concentration that is actually employed in the diluted sterilant composition used to sterilize articles. The “use” concentration is normally obtained by dilution of a concentrate having a higher content of peracetic acid. In one embodiment, the use concentration of peracetic acid is in the range from about 300 ppm to about 3000 ppm peracetic acid based on the sterilant composition used in sterilizing articles. In one embodiment, the use concentration is in the range from about 600 ppm to about 2400 ppm peracetic acid, based on the sterilant composition used in sterilizing articles. In one embodiment, the use concentration is in the range from about 800 ppm to about 1600 ppm peracetic acid, based on the sterilant composition used in sterilizing articles. To obtain this concentration, the commercially available peracetic acid, having a concentration of 37.5 wt % or 15 wt %, is used to prepare one or more intermediate mix including the various other ingredients that will be in the sterilant composition. Such additional ingredients include, for example, hydrogen peroxide, acetic acid, sulfuric acid, any (B) component additives such as an anticorrosive, a chelant and/or a buffer. The additional ingredients will be at similarly and appropriately diluted concentrations in the diluted sterilant composition, as described above for the “use” concentration of peracetic acid.

In one embodiment, the sterilant composition consists essentially of the components (A) and (B) as described above, in a non-aqueous solvent. In one embodiment, the sterilant composition consists essentially of the components (A) and (B) and includes a minor amount of water in the non-aqueous solvent.

As used herein, “a minor amount of water” means that the water present was not added as water, but is present as a component of one of the ingredients that also contains water, such as glacial acetic acid (e.g., 37% acetic acid in water), hydrogen peroxide, which may be obtained in concentrations as high as 98% hydrogen peroxide, but is more commonly obtained at lower concentrations.

In one embodiment, the sterilant composition consists of the components (A) and (B) in the non-aqueous solvent. In one embodiment, the sterilant composition consists of the components (A) and (B) and a minor amount of water in the non-aqueous solvent.

In one embodiment, component (A) may be limited to the ingredients necessary to form peracetic acid, in a non-aqueous solvent. In one embodiment, the ingredients necessary to form peracetic acid include acetic acid, hydrogen peroxide and sulfuric acid, in a non-aqueous solvent. In one embodiment, component (A) consists essentially of peracetic acid and the non-aqueous solvent. In one embodiment, component (A) consists essentially of the ingredients necessary to form peracetic acid in a non-aqueous solvent. In one embodiment, component (A) consists of peracetic acid and the non-aqueous solvent. In one embodiment, component (A) consists of the ingredients necessary to form peracetic acid in a non-aqueous solvent.

In embodiments in which the sterilant composition contains water and a pH can be determined, the sterilant composition may have a pH in the range from about 2 to about 11, or from about 5.5 to about 7. In some embodiments, it may not be possible to determine a pH, due to the low water content in the sterilant composition.

The sterilant composition may be referred to as a low-temperature sterilant composition, since it is effective at temperatures ranging from about 0° C. up to the standard high temperatures used in steam or high temperature sterilizations, but can be used in the range from about 0° C. to about 50° C., or from about 10° C. to about 30° C., or from about 20° C. to about 35° C.

This sterilant may be used in the sterilization of medical, dental, pharmaceutical, veterinary and mortuary devices, and the like, which cannot be subjected to the high temperatures required for steam sterilization.

In one embodiment, component (B) may be limited to three components, namely, a buffer, an anticorrosive agent and a chelator. In one embodiment, component (B) consists essentially of a buffer, an anticorrosive agent and a chelator. In one embodiment, component (B) consists of a buffer, an anticorrosive agent and a chelator.

The sterilant composition made from components (A) and (B) may be used in any process for sterilizing articles, including processes for sterilizing articles that cannot withstand the high temperatures required for steam sterilization. The articles that may be sterilized may include medical, dental, pharmaceutical, veterinary or mortuary instruments or devices (e.g., endoscopes), and the like. These may be made of a material comprising brass, copper, aluminum, stainless steel, carbon steel, plastic, glass, adhesive, or a combination of two or more thereof.

The process may be conducted in any suitable sterilization apparatus.

The articles may be sterilized using a sterilant composition formed from the non-aqueous solvent (C) and components (A) and (B), or from the non-aqueous solvent (C) and component (A) only. In one embodiment, component (A), and component (B), when present, are placed in a suitable chemical dispensing device and contacted with incoming non-aqueous solvent to form the sterilant composition.

### EXAMPLES

The following examples are based on 15 liters as the final volume of ‘Use Dilution’ used but, as will be understood, could be proportionately modified to any final volume desired. Here the term ‘Use Dilution’ represents the final mixture into which a device may be immersed for disinfection or sterilization. It could be any volume depending on the number and size of articles to be processed. For clarity, the bulk medium is referred to as the Diluent' and the sterilant is referred to as the ‘Active.’ Also, if and when another ingredient is added, e.g., an anticorrosive, buffer, chelator, etc., it is referred to as an ‘Additive.’ Incubation times may vary from 1 minute to 1 hour. Following the treatment, as an alternative to rinsing in the sterilizing apparatus, the device may be transferred to a rinsing agent in another container. Similar terms can be substituted as needed for brevity (e.g., Parts A, B, and C, respectively):

### Example 1

### Using Methanol and 35% PAA Concentrate With No Additives

To 14.875 L of methanol is added 125 ml of commercially available 35% peracetic acid (PAA) and mixed. The device is immersed in this use dilution at room temperature and incubated for 30 minutes with or without additional mixing. The use dilution is drained and the device is rinsed in methanol. After rinsing, the device is dried in air.

### Example 2

### Using Methanol and 15% PAA With No Additives

To 14.875 L of methanol is added 125 ml of commercially available 15% PAA (active) and mixed. The device is immersed in this use dilution at room temperature and incubated for 30 minutes with or without additional mixing. The use dilution is drained and the device is rinsed in isopropanol. After rinsing, the device is dried in air.

### Example 3

### 15% PAA Concentrate Diluted in Propylene Glycol With an Exemplary Anticorrosive Additive

To 14.875 L of propylene glycol is added 125 ml of commercially available 15% PAA, and 100 ppm of the anticorrosive hexapropyleneglycol cyclo-triphosphazene (HPGCP) and mixed. The device is immersed in this use dilution at room temperature and incubated for 60 minutes with or without additional mixing. The use dilution is drained and the device is rinsed in isopropanol. After rinsing, the device is dried in air.

### Example 4

### 15% PAA Concentrate Diluted in Ethylene Glycol With an Exemplary Anticorrosive Additive

To 14.875 L of ethylene glycol is added 125 ml of commercially available 15% PAA, and 10 ppm of the anticorrosive hexapropyleneglycol cyclo-triphosphazene (HPGCP) and mixed. The device is immersed in this use dilution at room temperature and incubated for 40 minutes with or without additional mixing. The use dilution is drained and the device is rinsed in isopropanol. After rinsing, the device is dried in air.

### Example 5

### Using Acetonitrile and 35% PAA Concentrate With No Additives

To 14.875 L of acetonitrile is added 125 ml of commercially available 35% PAA and mixed. The device is immersed in this use dilution at room temperature and incubated for 30 minutes without additional mixing. The use dilution is drained and the device is rinsed in ethanol. After rinsing, the device is dried in air.

### Example 6

### Using Tetrahydrofuran and 15% PAA With No Additives

To 14.875 L of tetrahydrofuran is added 125 ml of commercially available 15% PAA and mixed. The device is immersed in this use dilution at room temperature and incubated for 50 minutes without additional mixing. The use dilution is drained and the device is rinsed in methanol. After rinsing, the device is dried in air.

### Example 7

### 15% PAA Concentrate Diluted in Acetone With an Exemplary Anticorrosive Additive

To 14.875 L of acetone is added 125 ml of commercially available 15% PAA, and 100 ppm of the anticorrosive benzotriazole and mixed. The device is immersed in this use dilution at room temperature and incubated for 10 minutes with additional mixing. The use dilution is drained and the device is rinsed in acetone. After rinsing, the device is dried in air.

### Example 8

### 15% PAA Concentrate Diluted in Ethylene Glycol With an Exemplary Buffering Additive

To 14.875 L of ethylene glycol is added 125 ml of commercially available 15% PAA, and 10 ppm of the buffer disodium phosphate and mixed. The device is immersed in this use dilution at room temperature and incubated for 30 minutes with additional mixing. The use dilution is drained and the device is rinsed in n-propanol. After rinsing, the device is dried in air.

### Example 9

### 15% PAA Concentrate Diluted in Ethylene Glycol With an Exemplary Chelator Additive

To 14.875 L of ethylene glycol is added 125 ml of commercially available 15% PAA, and 10 ppm of the chelator EDTA and mixed. The device is immersed in this use dilution at room temperature and incubated for 5 minutes without additional mixing. The use dilution is drained and the device is rinsed in n-propanol. After rinsing, the device is dried in air.

Further EXAMPLES include the combination of 2 or more of the additives describe above. In even further EXAMPLES (as in the cases of EXAMPLES 1 and 2 above) the Diluent and Rinsing agent may be reversed in such a way as isopropanol is used as the diluent and the device can be rinsed in isopropanol or other suitable rinsing agent e.g. methanol. All EXAMPLES may be performed with or without applying heat in the range of 25 EC to 65 EC, or from 40 EC to 50 EC, or at the temperatures disclosed in the foregoing disclosures. Water may be used as the initial rinsing agent and followed by rinsing in a small amount of a non-aqueous solvent without compromising the essential non-aqueous nature of sterilization according to the present invention.

While the principles of the invention have been explained in relation to certain particular embodiments, these embodiments are provided for purposes of illustration. It is to be understood that various modifications thereof will become apparent to those skilled in the art upon reading the specification. Therefore, it is to be understood that the invention disclosed herein is intended to cover such modifications as fall within the scope of the appended claims. The scope of the invention is limited only by the scope of the claims.

1. A sterilant composition, consisting essentially of:
peracetic acid; and
a non-aqueous solvent.

2. The sterilant composition of claim 1, wherein the composition consists essentially of the peracetic acid, the non-aqueous solvent, and one or more of a buffer, an anticorrosive agent and a chelator.

3. The sterilant composition of claim 1 wherein the peracetic acid is a commercially available peracetic acid which consists essentially of the peracetic acid, acetic acid, hydrogen peroxide, sulfuric acid and a minor amount of water, the non-aqueous solvent present in a major amount in the sterilant composition.

4. The sterilant composition of claim 3 wherein the composition is free of separately added water.

5. The sterilant composition of claim 1 wherein the buffer comprises an alkali metal phosphate, an alkali metal carbonate, or a mixture thereof.

6. The sterilant composition of claim 1 wherein the anticorrosive agent comprises benzotriazole, tolyltriazole, a sodium salt of benzotriazole, a sodium salt of tolyltriazole, or a mixture of two or more thereof.

7. The sterilant composition of claim 1 wherein the chelator comprises ethylenediaminetetraacetic acid, hydroxyethylidenediphosphonic acid, a sodium salt of ethylenediaminetetraacetic acid, a sodium salt of hydroxyethylidenediphosphonic acid, or a mixture of two or more thereof.

8. The sterilant composition of claim 1 wherein the peracetic acid is dispersed in the non-aqueous solvent to form the sterilant composition, the concentration of peracetic acid in the non-aqueous solvent being in the range from about 0.1 to about 10 grams per liter.

9. The sterilant composition of claim 1 wherein the sterilant composition is free of water.

10. A process for sterilizing an article comprising contacting the article with the sterilant composition of claim 1.

11. The process of claim 10 wherein the article is made of a material comprising brass, copper, aluminum, stainless steel, carbon steel, plastic, glass, adhesive or a combination of two or more thereof.

12. The process of claim 10 wherein the article comprises a medical, dental, pharmaceutical, veterinary or mortuary instrument or device.

13. The process of claim 10 wherein the temperature of the sterilant composition is in the range from about 20 to about 80° C., or from about 40° C. to about 60° C.

14. The process of claim 10 wherein the exposure time of the article to the sterilant composition is in the range from about 0.5 to about 240 minutes, or from about 2 to about 60 minutes.
